(19) United States
(12) Patent Application Publication
(10) Pub. No.: US 2025/0009957 A1
(43) Pub. Date: Jan. 9, 2025

(54) NEGATIVE PRESSURE DRESSING

(71) Applicant: Aatru Medical, LLC, Cleveland, OH (US)

(72) Inventors: John Buan, Maple Grove, MN (US); Richard L. Middaugh, Rocky River, OH (US); Timothy Wojciechowski, Westlake, OH (US); Thomas E. Lash, Chardon, OH (US); Edward Armstrong, Chagrin Falls, OH (US)

(21) Appl. No.: 18/713,387

(22) PCT Filed: Dec. 2, 2022
(86) PCT No.: PCT/US2022/051661

§ 371 Date: May 24, 2024

Related U.S. Application Data
(60) Provisional application No. 63/285,662, filed on Dec. 3, 2021.

(51) Int. Cl. A61M 1/00; A61F 13/05

(52) U.S. Cl. CPC A61M 1/915; A61F 13/05; A61M 1/94; A61M 1/962; A61M 1/964

(57) ABSTRACT

A device for tissue treatment includes a drape configured to seal to skin of a patient to define a substantially air-tight enclosed volume around a tissue site. The drape includes an aperture extending through the drape and thus provides access to the enclosed volume. The device also includes a valve assembly attached to the drape, covering the aperture, and regulating a flow of air through the aperture. The valve assembly includes a base, an optional valve arranged in the base and configured to open to allow air to flow through the aperture, and to close to restrict air from flowing through the aperture, a top that is moveable with respect to the base between an opened position and a closed position, and a seal arranged on the top, and scaling to the base over the aperture and optional valve when the top is in the closed position.

### CROSS REFERENCE TO RELATED APPLICATIONS

This application claims the benefit of U.S. Provisional Patent Application Ser. No. 63/285,662 filed Dec. 3, 2021, which is incorporated herein by reference.

### BACKGROUND

Negative pressure therapy is a treatment that utilizes negative pressure for skin treatments and restorative purposes. Negative pressure is a term used to describe a pressure that is below normal atmospheric pressure. Negative pressure therapy is utilized for several sites on the skin, such as a wound or an incision Furthermore, negative pressure therapy is useful to manage wounds with complex healing concerns. Additionally, negative pressure therapy could also be used for cosmetic purposes like removing wrinkles.

Generally, negative pressure therapy is achieved by maintaining a reduced pressure beneath a dressing at a dressing site.

### SUMMARY

In an embodiment, a device for tissue treatment includes a drape and a valve assembly. The drape is configured to seal to skin of a patient to define a substantially air-tight enclosed volume around a tissue site, and includes an aperture extending through the drape and thus providing access to the enclosed volume. The valve assembly is attached to the drape, covers the aperture, and regulates a flow of air through the aperture. The valve assembly includes a base, a top that is moveable with respect to the base between an opened position and a closed position, and a seal arranged on the top and sealing to the base over the aperture when the top is in the closed position.

In another embodiment, a system for tissue treatment further includes a mechanical pump that when activated and engaged to a flat top surface of the base when the top is in the opened position, thereby removes air from the enclosed volume.

### BRIEF DESCRIPTION OF THE DRAWINGS

FIG. 1 is a perspective view of a dressing with a valve assembly according to the present subject matter.

FIG. 2 is a perspective view of the dressing of FIG. 1 with an open valve assembly being engaged by a pump assembly according to the present subject matter.

FIG. 3 is a perspective cross sectional view of the dressing of FIG. 1.

FIG. 4 is a perspective view of a dressing with a valve assembly according to the present subject matter.

FIG. 5 is a perspective view of the dressing of FIG. 4 with an open valve assembly being engaged by a pump assembly according to the present subject matter.

FIG. 6 is a perspective cross sectional view of the dressing of FIG. 4.

FIG. 7 is a perspective view of a dressing with a valve assembly according to the present subject matter.

FIG. 8 is a perspective view of the dressing of FIG. 7 with an open valve assembly being engaged by a pump assembly according to the present subject matter.

FIG. 9 is a perspective cross sectional view of the dressing of FIG. 7.

FIG. 10 is a perspective view of a dressing with a valve assembly and a cap assembly according to the present subject matter.

FIG. 11 is a perspective view of a cap for use in a cap assembly according to the present subject matter.

FIG. 12 is a cross section view of the cap assembly with an intact seal according to the present subject matter.

FIG. 13 is a cross section view of the cap assembly of FIG. 12 with a broken seal.

FIG. 14 is a perspective view of a dressing according to the present subject matter.

FIG. 15 is a cross section view of a dressing with a reactor in an inactivated state according to the present subject matter.

FIG. 16 is a cross section view of the dressing of FIG. 15 with the reactor being activated and an open second aperture.

FIG. 17 is a cross section view of the dressing of FIGS. 15 and 16 with the reactor being activated and a sealed second aperture.

### DETAILED DESCRIPTION

With reference to the figures, a negative pressure dressing 2 (i.e. a negative pressure dressing) can be applied to skin for tissue treatment, including for negative pressure therapy of the tissue. The dressing 2 includes a drape/housing 4, which is placed over a tissue site (i.e. skin) of a patient so as to define a substantially air-tight enclosed volume around the tissue site and beneath the drape 4. As used herein, “substantially air-tight” means that there is no bulk air flow or viscous air flow between the atmosphere and the enclosed volume, where the bulk or viscous air flow is the flow of air that produces an equal effect on the amount of each of the different components of air (i.e. different gasses in air such as O₂ and N₂). In other words, bulk air flow is a movement of air between the atmosphere and the enclosed volume that does not change the percentages of the different gases in air in the enclosed volume, and this bulk air flow is prevented when the drape is substantially air-tight. This meaning for “substantially air-tight” is in contrast to the “permeation” of gases between the atmosphere and the enclosed volume, which permeation produces, not equal but, different effects on the amount of each of the different gasses in air. In other words, permeation is a movement of gasses between the atmosphere and the enclosed volume that does/may change the percentages of the different gases in air in the enclosed volume, and this permeation is not necessarily prevented when the drape 4 forms a substantially air-tight enclosed volume over the tissue site.

The tissue site covered by the drape 4 may be, but is not limited to, a wound, an incision, or skin where there is no wound or incision. The dressing 2 can be positioned at the tissue site to enhance tissue treatment including, but not limited to, therapeutic treatments including wound healing and other medical treatments, cosmetic treatments including the reduction of skin wrinkles, and/or other skin issues or maladies.

### Drape

The drape 4 may be a rigid drape or a flexible drape. In an non-limiting embodiment, the drape 4 is a flexible drape. The drape 4, in the form of a film, may be made from a flexible material including a thin, flexible, elastomeric film. Examples of such materials include polyurethane or polyethylene films. If oxygen is removed from the enclosed volume under the drape, such as by use of an oxygen scavenger (as will be discussed in more detail herein), the drape 4 may be capable of maintaining a low-oxygen environment (i.e. less than 21% as in ambient air) in the enclosed volume underneath the drape 4 with use of such an oxygen scavenger. The removal of oxygen gas from the enclosed volume may also create a low-pressure environment in the enclosed volume. For this, the dressing 2 may include internal components arranged under the flexible drape that have a resistance to compression, thereby creating enough volume under the drape to produce and maintain a negative pressure under the drape, e.g. between −25 mmHg and −160 mmHg, as a result of oxygen gas being removed by the oxygen scavenger.

The thin film from which the drape 4 is made can be substantially impermeable to liquids but somewhat permeable to water vapor, while still being capable of maintaining a low-oxygen environment in the enclosed volume underneath the drape 4 during use of an oxygen scavenger (e.g. a reactor 90 as discussed herein). For example, the thin film material from which the drape 4 could be made include polyurethane or other semi-permeable material such as that sold under the Tegaderm® brand or 9834 TPU tape available from 3M. Similar films are also available from other manufacturers. Even though the film from which the drape 4 is made may have a water vapor transmission rate of about 836 g/m²/day or more, these films are still capable of maintaining negative pressure and/or a low-oxygen environment for one or more hours or days in the enclosed volume underneath the drape 4 when an appropriate seal is made around the periphery of a tissue site between the drape 4 and the tissue.

The dressing 2 may include a gasket 6 at a bottom surface 8 of the drape 4 for sealing the drape 4 to the skin around the tissue site in a substantially air-tight manner, thus defining the enclosed volume around the tissue site. The gasket 6 can be a silicone gel. Other types of gasket materials may be employed, such as a hydrogel. The gasket 6 may include a backing film, on which the silicone gel, hydrogel, or other sealing material of the gasket 6 is applied. The backing film can be a polyurethane, polyethylene, polypropylene, or co-polyester film, and can be brought into contact with an adhesive layer applied to the bottom surface 8 of the drape 4 in order to fix the silicone gel gasket 6 to the drape 4. The gasket 6 may be robust enough for maintaining a therapeutic negative pressure in the enclosed volume around a tissue site, which therapeutic negative pressure is between −25 and −160 mmHg with respect to atmospheric pressure. The gasket 6 may be robust enough for maintaining a pressure in the enclosed volume around a tissue site from +150 mmHg to −160 mmHg, which pressure can be adjusted as desired, including by the use of the mechanical pump 86 and/or reactor 90.

The dressing 2 may also include a tissue site contacting layer 10 arranged within the enclosed volume and within a perimeter formed by the gasket 6, e.g. the gasket 6 may be a continuous annulus or have a general annular shape, and thus radially surround the tissue site contacting layer 10 by forming a perimeter around the tissue site contacting layer 10. The tissue site contacting layer 10 may be affixed to the bottom surface 8 of the drape 4 by an adhesive layer on the bottom surface 8 of the drape 4. The tissue site contacting layer 10 may include an absorbent material, which can be made from super absorbent polymers, absorbent beads, foams, or natural absorbents. For example, the absorbent material can be a hydroactive non-woven wound pad, such as that available from Freudenberg Performance Materials, which chemically absorbs exudate and precludes the exudate from passing through the absorbent material when not fully saturated. In a non-limiting embodiment, the absorbent material may also be an easily compressible porous material to allow the enclosed volume under the drape 4 to decrease in size as needed to maintain internal pressure close to ambient pressure. In another non-limiting embodiment, an internal component arranged under the drape 4, may have a resistance to compression and thus maintain the size of the enclosed volume under the drape 4 so that a removal of air (or a selected gas found in air) from the enclosed volume results in a decrease in pressure in the enclosed volume. The tissue site contacting layer 10 may be designed to be relatively non-compressible, so as to maintain a size of the enclosed volume, e.g. even under a reduced pressure inside the enclosed volume.

The tissue site contacting layer 10 can also be designed to allow for a bulk flow of air through it as well as for contacting a wound, and can be made of an elastomeric material, such as a polymeric material that has rubber-like properties. The elastomeric material can be a thin, flexible elastomeric film. Some examples of such material include a silver coated nylon, a perforated silicone mesh, or other materials that will not stick to the tissue site of a patient. If desired, antibacterial or antimicrobial materials may be deposited on/in the tissue site contacting layer 10.

The dressing 2 may include the adhesive layer applied to the bottom surface 8 of the drape 4. The adhesive layer (not shown) may be applied by flood coating to cover the entire bottom surface 8 of the drape 4, or applied to select portions of the bottom surface 8. The adhesive layer may be used to adhere the tissue site contacting layer 10 to the bottom surface 8 of the drape 4. The gasket 6 or its backing film may also be applied to the adhesive layer, so as to leave a margin of the adhesive layer in a flange area 12 that annularly surrounds the gasket 6, which flange area 12 at the margin of the dressing 2 may be used to adhere the dressing 2 to skin. Alternatively, the gasket 6 and the tissue site contacting layer 10 may be applied directly to the bottom surface 8 of the drape 4, with a different adhesive(s), and the adhesive layer applied to the bottom surface 8 may be applied as an annulus around the perimeter of the gasket 6. The adhesive layer applied to the bottom surface 8 of the drape 4 may be a pressure-sensitive acrylic-based adhesive. Other types of adhesives could be used, however, a pressure-sensitive acrylic-based adhesive is known to provide strong initial tack to skin that can last for a relatively long time, for example a few days, when in contact with the skin. The pressure-sensitive acrylic-based adhesive can be applied over an entirety of the of the bottom surface 8 of the drape 4, or only to select portions of the bottom surface 8.

Also, it is well known that typical adhesives, in particular pressure-sensitive acrylics such as those that can be included on the bottom surface 8 adhering the drape 4 to the patient's skin, do not form air-tight seals with skin. Thus, the drape 4 may be well adhered to the skin by the adhesive layer, but may still allow ambient air to enter between the drape 4 and the skin and into the enclosed volume beneath the drape 4. For example, hair follicles and other irregularities on the skin may prohibit an adhesive from forming an adequate air seal between the drape 4 and the skin, thus preventing the enclosed volume from reaching what is often considered to be a therapeutic negative pressure, which is typically between −25 and −160 mmHg, as the selected gas is being removed from the volume by the reactor 90. These “leaks” of air flowing past the adhesive layer on the bottom surface 8 of the drape 4 may be overcome either by including the gasket 6, which forms a substantially air-tight seal between the dressing 2 and the skin, or when the reactor 90 has sufficient capacity to maintain the low-oxygen environment even when these leaks exist. In other circumstances not involving adhering to skin, it is noted that typical adhesives are able to form air-tight seals between two polymer materials. Therefore, typical adhesive may be used to form air-tight seals (discussed herein) between various polymer components of the dressing 2, such as between the drape 4 and the valve assembly 20.

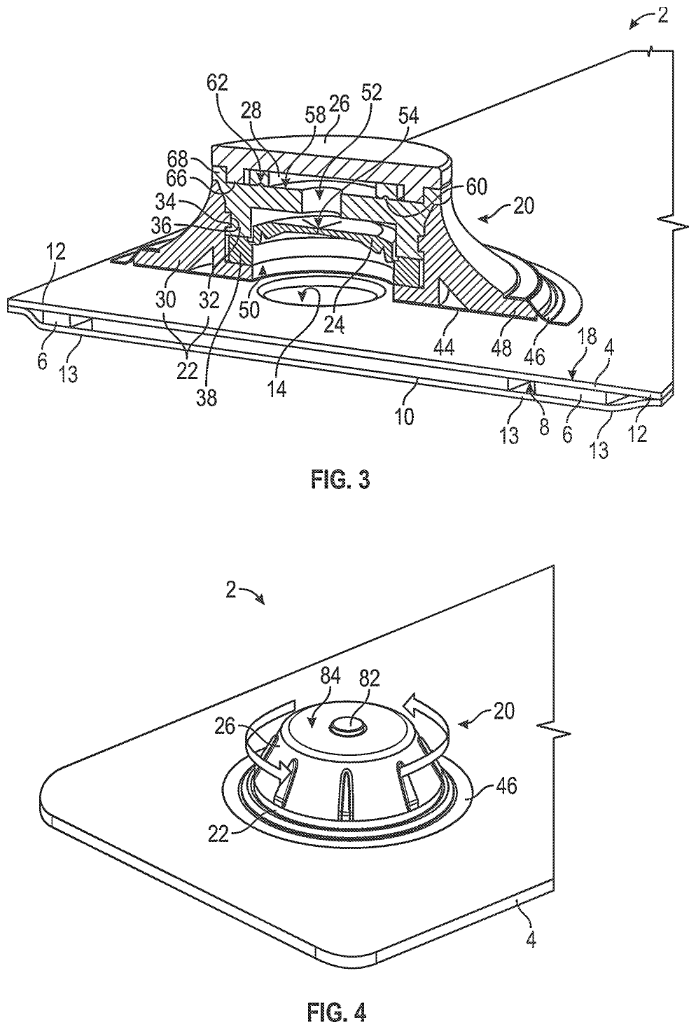

The dressing 2 may include a release liner 13 (FIGS. 3, 6, 9) disposed on the bottom of the dressing 2 to cover the exposed adhesive layer in the flange area 12, the gasket 6, and the optional tissue site contacting layer 10, and to inhibit contamination of these components. The release liner 13 is removed before the dressing 2 is applied to the tissue site. When the release liner 13 is removed, the gasket 6, the adhesive on the bottom surface 8 in the flange area 12 surrounding the gasket 6, and the tissue site contacting layer 10, are exposed. As the dressing 2 is placed on the patient's skin, the adhesive, which can be an acrylic-based adhesive in the flange area 12, secures the drape 4 to the patient's skin around the tissue site.

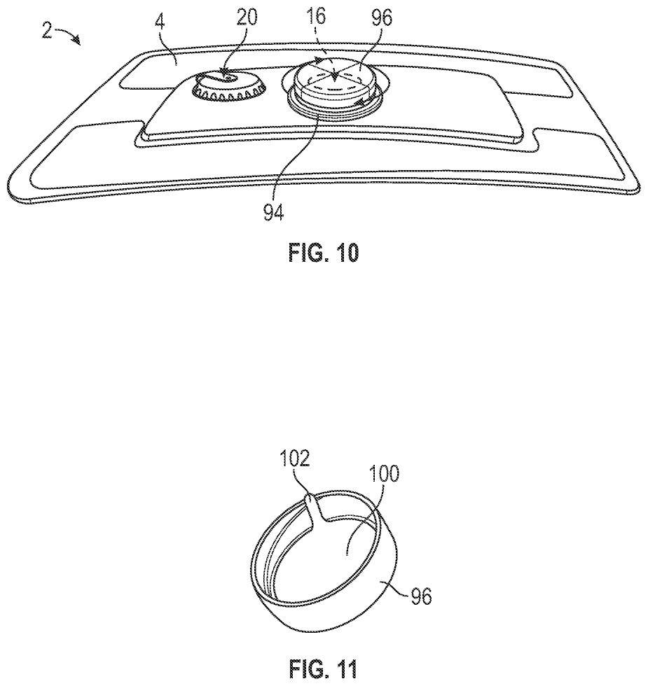

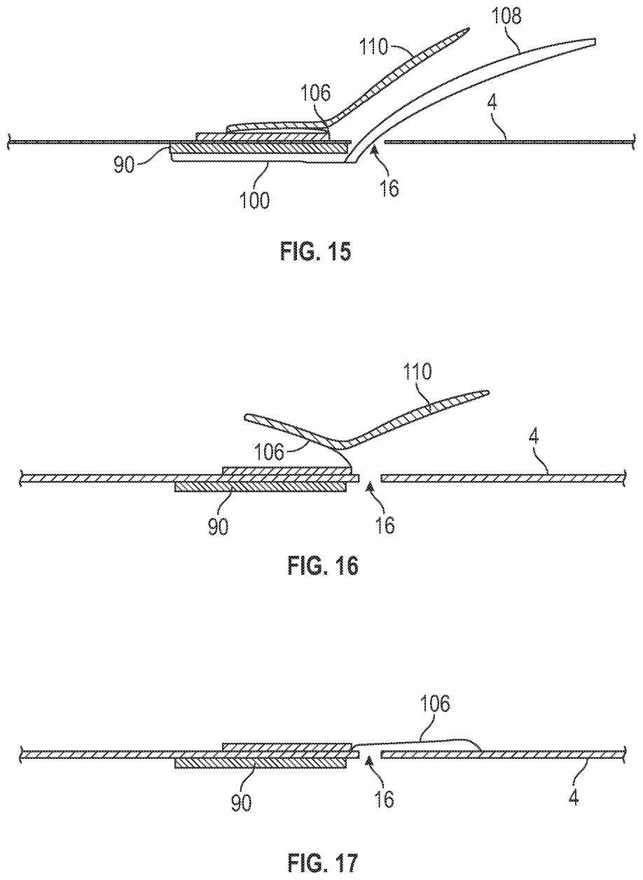

The drape 4 includes a first aperture 14, and optionally a second aperture 16 (FIGS. 10, 14, and 15-17). The first and second apertures 14, 16 are through holes that extend completely through the thickness of the drape 4 from the top surface 18 of the drape 4 to the bottom surface 8 of the drape 4, thus providing access to the enclosed volume under the drape 4. The first and second apertures 14, 16 are arranged inside the perimeter formed by the gasket 6, and thus provide access into the enclosed volume.

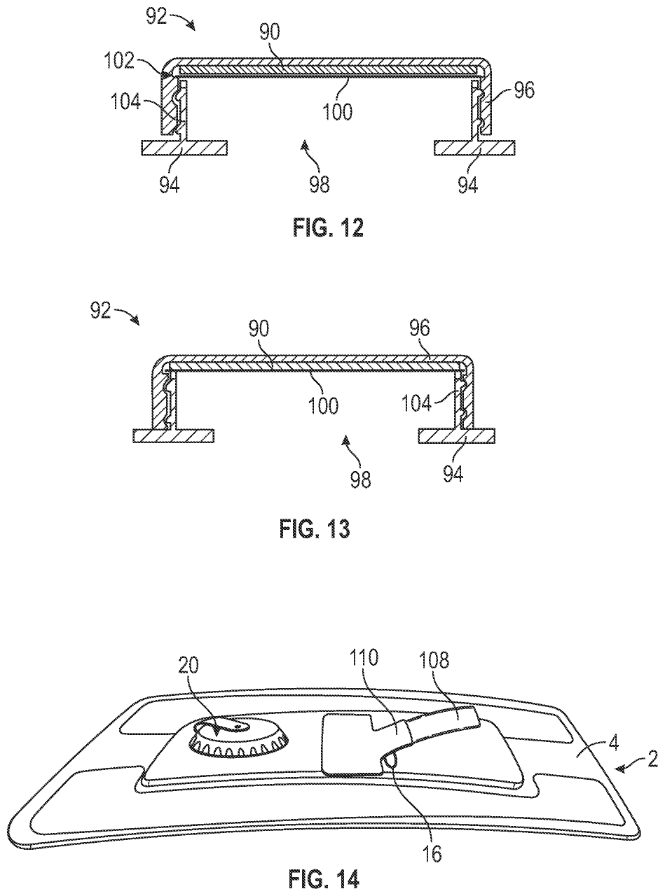

The drape 4 can be made from a material that is air impermeable to bulk or viscous air flow so that air is precluded or greatly inhibited from entering into the enclosed volume by bulk or viscous flow. If included, a reactor 90 (FIGS. 12, 13, 15-17) can consume a selected gas within the enclosed volume, thus reducing the partial pressure of the consumed gas within the enclosed volume, and also possibly reducing the total gas pressure within the enclosed volume when the drape 4 is rigid or otherwise has internal components resistant to compression. The drape 4 could also be made from materials that allow for some level of permeation of gases through the drape 4. The drape 4 can be formed of a material that is at least partially gas permeable for certain gasses (e.g., permeable to oxygen or nitrogen gases) to allow the gas(es) to permeate between the tissue site in the enclosed volume and the atmosphere.

### Valve Assembly

The dressing 2 includes a valve assembly 20 sealed to the top surface 18 of the drape 4. The valve assembly 20 can be sealed over the first aperture 14 in a substantially air-tight manner to block air from passing through the first aperture 14, and for this purpose, an adhesive can be used. The valve assembly 20 includes a base 22, a valve 24, a top 26, and one or more optional seals 28.

### Base

The base 22 may include a lower base portion 30 and an upper base portion 32. The lower base portion 30 is arranged under the upper base portion 32 and adjacent the top surface 18 of the drape 4. The upper base portion 32 is connected to the lower base portion 30.

The valve 24 is arranged (e.g. held, mounted, or clamped) between the lower base portion 30 and the upper base portion 32. The lower base portion 30 and the upper base portion 32 may be held together, and thus clamp the valve 24 between them, by an interlocking joint, an adhesive joint, a welded joint, a friction fit joint, or otherwise.

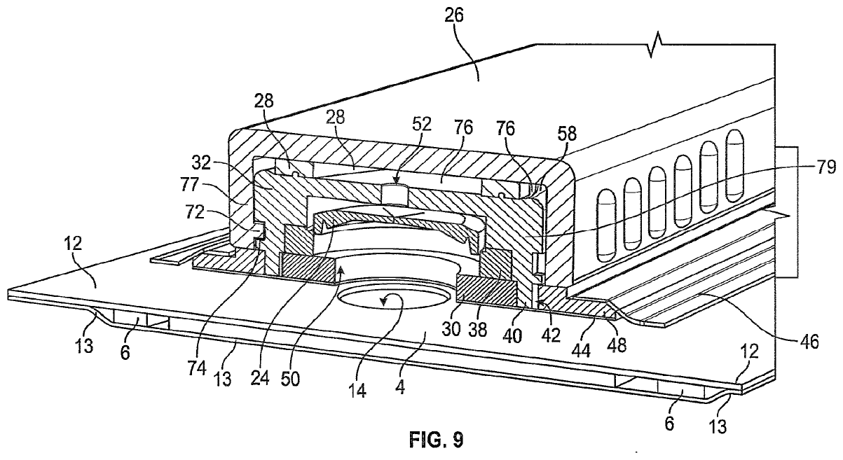

As shown in FIG. 3, an interlocking joint in the form of circumferential projection 34 on the lower base portion 30 and a corresponding circumferential groove 36 on the upper base portion 32 are mated to lock the lower and upper base portions 30, 32 together, and thus hold a peripheral edge 38 of the valve 24 between the lower base portion 30 and the upper base portion 32. As shown in FIGS. 6 and 9, a leg 40 of the upper base portion 32 fits into a slot 42 of the lower base portion 30, and these may be secured together as desired, e.g. by an adhesive, to hold the edge 38 of the valve 24 between the lower base portion 30 and the upper base portion 32. Other connection arrangements can be used, and the base 22 can also be a one-part construction.

The lower base portion 30 is sealed to the top surface 18 of the drape 4 in a substantially air-tight manner completely circumferentially around the first aperture 14. This may be accomplished by using a base adhesive 44 and an adhesive tape 46, either one or both providing adhesion and/or the substantially air-tight seal. The base adhesive 44 is arranged between the lower base portion 30 and the drape 4, and adheres the lower base portion 30 to the drape 4. The adhesive tape 46 adheres to the top surface 18 of the drape 4 and overlies part of the lower base portion 30, e.g. adheres to an upper surface of a lower perimeter/edge 48 of the lower base portion 30 around an entire perimeter of the base 22.

The lower base portion 30 includes a through hole 50. The lower base portion 30 is generally aligned over the first aperture 14 so that the lower base portion completely radially surrounds the first aperture 14 and seals to the top surface 18 of the drape 4 completely around the perimeter of the first aperture 14. The lower base portion 30 is attached to the drape 4 such that the through hole 50 is in fluid communication with the first aperture 14, and optionally concentrically with the first aperture 14.

The upper base portion 32 includes one or more through holes 52. The upper base portion 32 is generally aligned over the first aperture 14 such that when the valve 24 is open, the through hole(s) 52 is in fluid communication with the first aperture 14 and with the through hole 50 of the lower base portion 30 via the opened valve 24, to thereby create a channel in the valve assembly 20 that allows a bulk flow of air between the enclosed volume and the atmosphere exterior of the dressing 2. The through hole(s) 52 in the upper base portion 32 and the through hole 50 in the lower base portion 30 together define a through hole of the base 22.

### Valve

The valve 24 may provide a primary seal for the first aperture 14 that is substantially air-tight. The primary seal provided by the valve 24 may not be substantially air-tight however, and thus may be supplemented by a secondary seal provided by the top 26, as discussed in detail herein. The valve 24 may be biased so that is normally closed, but may operate to open under a pressure condition, i.e. where there is a pressure differential across the valve 24 that exceeds a predetermined threshold(s). The valve 24 is configured to open to allow a bulk flow of air through the first aperture 14, and to close to restrict a bulk flow of air through the first aperture 14.

The valve 24 may be a valve as described in U.S. Pat. No. 5,213,236, which is incorporated by reference herein. The valve may include intersecting linear slits 54 defining flaps, by which the valve 24 opens to define an opening near a center of the valve 24 when subjected to a pressure condition. The opening in the valve 24 may involve the flaps separating from one another at the slits 54 to create an opening that is fluid communication with the through holes 50, 52 and with the first aperture 14. When the flaps are not separated from each other, then the valve 24 is closed and does not define an opening. The valve 24 can also have just a single slit rather than two slits 54 defining flaps. Additionally, the valve 24 could also be one-way valve, without ability to admit ambient air into the enclosed volume.

The valve 24 can be configured to open to allow a bulk flow of air out of the enclosed volume when a mechanical pump is used to suck air out of the enclosed volume through the valve assembly 20. The valve 24 can also be configured to open and allow air into the enclosed volume beneath the drape 4 to avoid a pressure difference between the enclosed volume and the ambient pressure that is greater than desired. For example, the valve 24 can be configured to open to allow air into the enclosed volume when the pressure differential between the enclosed volume and the ambient pressure outside the enclosed volume is more than 150 mmHg. This may occur if the mechanical pump assembly 86 has reduced the pressure in the enclosed volume below that required for the intended therapeutic condition. As such, when the valve 24 is open, air may then enter through the valve 24 and the valve assembly 20, and into the enclosed volume beneath the drape 4, thus raising the air pressure in the enclosed volume towards the therapeutic range. The valve 24 can be configured to close again when the pressure differential between the enclosed volume and ambient pressure outside the enclosed volume is within the desired range. In this way, the enclosed volume can equilibrate dynamically to the desired pressure range.

### Top

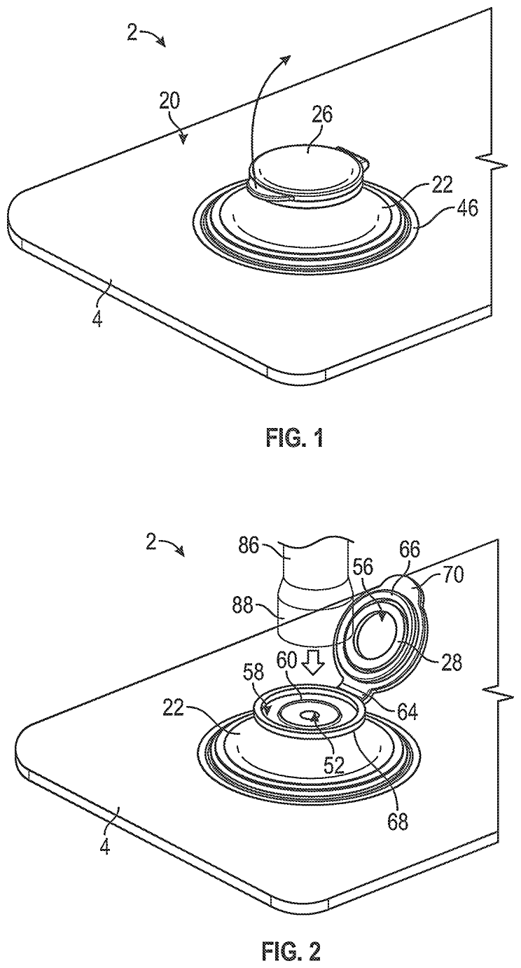

The top 26 of the valve assembly 20 is used to cover over the other components of the valve assembly 20, and optionally to provide a secondary seal for the first aperture 14 that may be substantially air-tight, and which may supplement the primary seal provided by the valve 24. The top 26 is moveable with respect to the base 22 between an opened position (FIGS. 2, 5, 8) and a closed position (FIGS. 1, 3, 4, 6, 7, 9).

In order to create the secondary seal for the first aperture 14, the top 26 may include one or more seals 28 on its bottom surface 56, each of which may be an annular seal. When the top 26 is in the closed position, the seal(s) 28 contact, and thus seal to, an upper surface 58 of the upper base portion 32, over the valve 24, and around the through hole(s) 52 of the upper base portion 32 to provide the secondary seal for the first aperture 14. The seal(s) 28 may contact an annular ridge 60 in the upper surface 58 of the base 22, which annular ridge 60 may temporarily create or mate with an annular depression 62 in the seal(s) 28. In one non-limiting embodiment, the valve 24 is not included, and the seal(s) 28 and base adhesive 44 and adhesive tape 46 create the only seals of the first aperture 14.

When the top 26 is in the opened position, the upper surface 58 of the base 22 and the through hole(s) 52 of the upper base portion 32 are exposed and not covered by the top 26. When the top is in the closed position, the upper surface 58 of the base 22 and the through hole(s) 52 of the upper base portion 32 are covered by the top 26, and the seal(s) 28 create the secondary seal for the first aperture 14 by sealing to the upper surface 58 of the base 22.

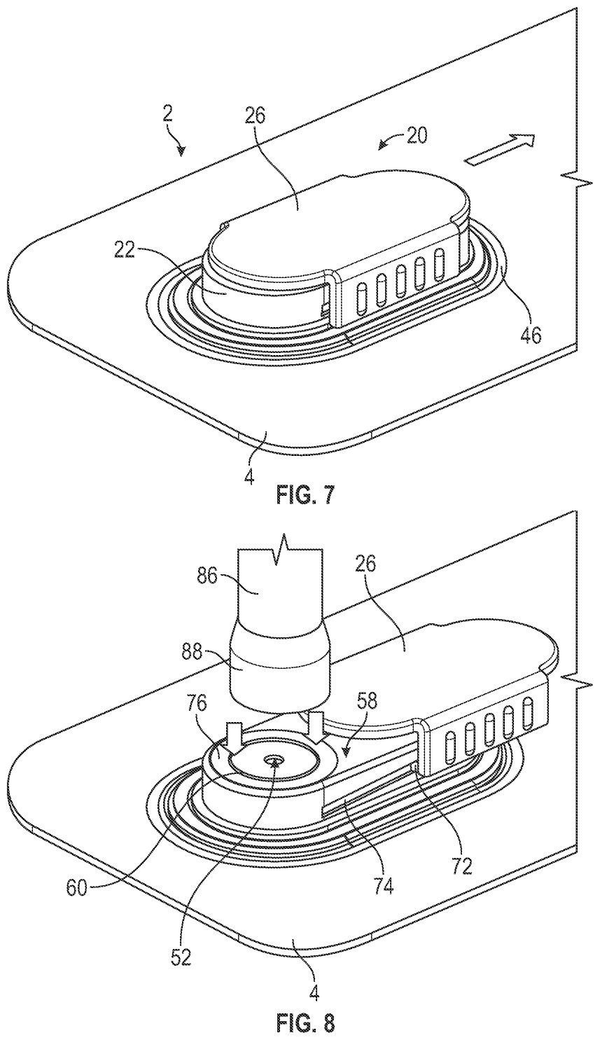

The top 26 may be connected with the base 22 by a moveable joint so that the top 26 can move relative to the base 22 between the opened position and the closed position. The moveable joint between the top 26 and the base 22 may include hinge joint (FIGS. 1-3), a threaded joint (FIGS. 4-6), a slide joint (FIGS. 7-9), or other types of moveable joints.

Where the valve assembly 20 includes the hinge joint (FIGS. 1-3), the top 26 and upper base portion 32 may be a one-piece construction including a hinge 64, where the hinge 64 is arranged between and connects the top 26 and the upper base portion 32. The hinge 64 may also be a distinct component from the top 26 and upper base portion 32. In this configuration, the top 26 may be moved relative to the base 22 between the opened position and the closed position by swinging/rotating the top 26 about the hinge 64 (see arrow in FIG. 1 and change in position between FIGS. 1 and 2).

In this non-limiting embodiment (or others), the top 26 may include a first annular projection 66 that extends from the bottom surface 56 of the top 26. When the top 26 is in the closed position, the first annular projection 66 aligns the top 26 to the base 22 by fitting radially inside a second annular projection 68 on the base 22 that extends from the upper surface 58 of the base 22 (See FIG. 4). The seal(s) 28 on the top 26 may be arranged radially inside the first annular projection 66. The top 26 may also include a tab 70 for making it easier to move the top 26 from the closed position to the opened position by hand. The top 26 may include only one seal 28 and the upper base portion 32 may include only one through hole 52.

Where the valve assembly 20 includes a threaded joint (FIGS. 4-6), the top 26 and the upper base portion 32 may be distinct components, and may be joined by mating threads on the top 26 and the upper base portion 32. The top 26 may have laterally projecting threads 78 engaging with laterally indented threads 81 of the upper base portion 32, or vice versa. In this configuration, the top 26 may be moved relative to the base 22 between the opened position and the closed position by turning or rotating the top 26 relative to the base 22 (See arrows in FIG. 4). The opened position and the closed position may be separated by a quarter turn or rotation of the top 26, or more or less, relative to the base 22. The top 26 may include two seals 28, and the upper base portion 32 may include at least two through holes 52 or more.

When the top 26 is in the closed position, the two seals 28 engage the flat upper surface 58 of the base 22 or to an annular ridge 60 of the upper surface 58 of the base 22 to seal to the upper surface 58 of the base 22 (See FIG. 6). The at least two through holes 52 in the upper base portion 32 are arranged radially between the two seals 28 so as to seal the through holes 52. The top 26 may be moved from the closed position to the opened position by turning the top 26 relative to the base 22 in one direction (e.g. counter clockwise), which moves the top 26 up away from the upper base portion 32, thus moving the seals 28 up away from the upper surface 58 of the base 22 such that the seals 28 no longer touch or seal to the upper surface 58 of the base 22. This unsealing of the through holes 52 allows a bulk flow of air past the seals 28, through the through holes 52, and either out or in through an opening 80 in the top 26.

The top may be moved from the opened position to the closed position by turning the top 26 relative to the base 22 in the other direction (e.g. clockwise), which moves the top 26 down toward the upper base portion 32, thus moving the seals 28 down toward the upper surface 58 of the base 22 such that the seals 28 touch and seal to the upper surface 58 of the base 22. This sealing of the through holes 52 inhibits a bulk flow of air past the seals 28, through the through holes 52, and either out or in through the opening 80 in the top 26.

The upper base portion 32 may include a projection 82. When the top 26 is in the closed position, the projection 82 sticks up through the opening 80 in the top 26 and above a top surface 84 of the top 26 (See FIG. 6). The projection 82 is elevated above the top 26 when the top 26 is in the closed position, and thus provides a visual indication of the closed position.

Where the valve assembly 20 includes the slide joint (FIGS. 7-9), the top 26 and the upper base portion 32 may be distinct components, and may be joined by lateral projections 72 of the top 26 engaging into lateral indentations 74 on the upper base portion 32. The lateral projections 72 may extend laterally from vertical side walls 77 of the top 26. The lateral indentations 74 may extend laterally into vertical side walls 79 of the upper base portion 32. In this configuration, the top 26 may be moved relative to the base 22 between the opened position and the closed position by sliding the top 26 relative to the base 22 in a lateral direction (See arrow in FIG. 7 and change of position between FIGS. 7 and 8), thus exposing the through hole 52 in the upper base portion 32. The lateral projections 72 can be slidably arranged in the lateral indentations 74.

The lateral indentations 74 may be sloped (See FIG. 8), rather than being level, such that as the lateral indentations 74 extend further away from the through hole 52 in the upper base portion 32, the lateral indentations 74 extend closer to the upper surface 58 of the base 22 (i.e. have a positive slope). This sloped configuration for the lateral indentations 74 allows the top 26 to be moved closer to the upper surface 58 of the base 22 as the top 26 is moved from the opened position to the closed position, and thus assists in the seal 28 to press against the upper surface 58 of the base 22 and seal to the upper surface 58 of the base 22. This also allows the top 26 to be moved further away from the upper surface 58 of the base 22 as the top 26 is moved from the closed position to the opened position, and thus assists in the seal 28 not pressing against the upper surface 58 of the base 22 and not sealing to the upper surface 58 of the base 22 and allowing easier lateral movement of the top 26 to the opened position.

The upper surface 58 of the base 22 may include a depression 76, e.g. a circular depression, extending into the upper surface 58 of the base 22. When the top 26 is in the closed position, the seal 28 may be arranged entirely radially inside the depression 76 (See FIG. 16). When the top 26 is in the opened position, the seal 28 may be arranged at least partially radially outside (e.g. entirely radially outside) the circular depression 76. The top 26 may include only one seal 28 and the upper base portion 32 may include only one through hole 52. A seal may still be formed between the seal 28 and the upper surface 58 of the base 22 by the seal 28 being compressed via movement of the top 26 along the sloped lateral indentations 74.

### Mechanical Pump

The dressing 2 may be included as a part of a system for tissue treatment, along with a mechanical pump assembly 86, which is used to remove air from the enclosed volume through the first aperture 14 and through the valve assembly 20.

The mechanical pump assembly 86 is not particularly limited, and may include a powered or manual pump (not shown), and a head 88 for connecting with the valve assembly 20 for removing air from the enclosed volume. When the mechanical pump assembly 86 is connected to the dressing 2, the mechanical pump assembly 86 is in fluid communication with the enclosed volume via the valve assembly 20.

Actuation of the pump draws air from the head 88 connected to the valve assembly 20 when the top 26 is in the opened position. Such drawing of air by the pump causes a pressure differential at the valve 24, which causes the valve 24 to open. Air in the enclosed volume can then be drawn through the first aperture 14, through the through hole 50 of the lower base portion 30, through the opened valve 24, through the one or more through holes 52 of the upper base portion 32, and into the mechanical pump assembly 86 via the head 88. When this is done, the enclosed volume experiences a reduction in air pressure therein. Further, the sealing of the dressing 2 against the skin can be checked at this point because the drape 4, if it were flexible, would be drawn toward the skin as a result of this reduction in air pressure in the enclosed volume. The head 88 can then be removed from the valve assembly 20, which allows the valve 24 to return to its biased closed configuration, which would inhibit a bulk flow of air into or out of the enclosed volume.

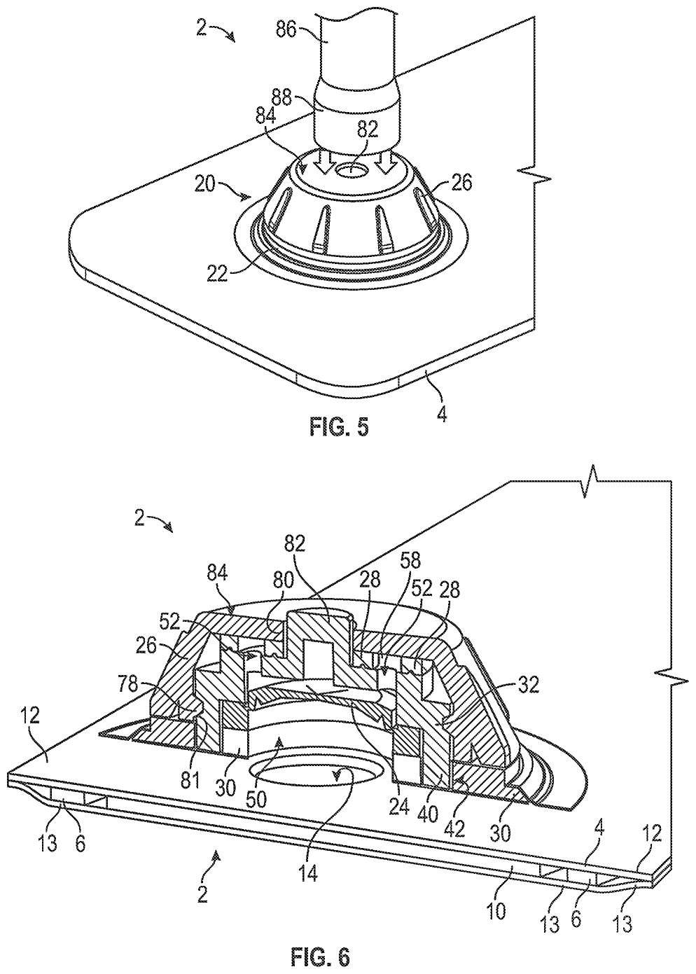

The head 88 is configured to connect to the valve assembly 20 by engaging either the upper surface 58 of the base 22 (FIGS. 2, 8, when the top 26 is in the opened position), or the upper surface of the top 26 (FIG. 5). The mechanical pump assembly 86 is in fluid communication with the enclosed volume when the top 26 is in the opened position, when the valve 24 is opened, and when the head 88 is connected to the valve assembly 20. The head 88 may include a seal, similar to the seal 28 on the top 26, for producing a seal and allowing for the suction of air from the enclosed volume, through the valve assembly 20, and into the mechanical pump assembly 86. The removal of air from the enclosed volume may decrease the pressure inside the enclosed volume.

When the head 88 is connected to the valve assembly 20, the top 26 is in the opened position, and the mechanical pump assembly 86 is activated, then a vacuum pressure is created on the upper side of the valve 24. This causes the valve 24 to move from its biased closed position and to an open position (e.g. by the flaps separating from each other to form an opening in the valve 24, or other process of opening the valve 24), thus creating a bulk flow path from the enclosed volume, through the first aperture 14, through the valve assembly 20, and into the mechanical pump assembly 86. Air can then be sucked through this bulk flow path from the enclosed volume to the mechanical pump assembly 86 to thereby remove air from the enclosed volume. This may create a low-pressure condition (i.e. less than atmospheric pressure) inside the enclosed volume.

Once the mechanical pump assembly 86 is deactivated or detached from the valve assembly 20, then the vacuum pressure is relieved from the upper side of the valve 24, which allows the valve 24 to move back towards its biased closed position, e.g. by the flaps coming back into contact with one another and thus cutting off the bulk flow path that was previously created. Air is thus inhibited from flowing back into the enclosed volume.

### Reactor

The system for tissue treatment may also include a reactor 90 (FIGS. 10-17) along with the valve assembly 20. The reactor 90 is configured to react with (e.g. consume) a selected gas (e.g., nitrogen, oxygen, carbon dioxide) found in air. In a non-limiting embodiment, the reactor 90 may react with oxygen, and thus is referred to herein as an oxygen scavenger. As the reactor 90 consumes the selected gas within the enclosed volume and the valve assembly 20 seals off the first aperture 14, the amount of oxygen and the total gas pressure within the enclosed volume may be reduced. For example, where the reactor 90 is an oxygen scavenger and thus consumes oxygen in the enclosed volume, and where the reactor 90 is under a rigid drape 4 or is under a flexible drape 4 that is supported by internal components that are resistant to compression, then there can be an approximate 20% reduction in the total gas pressure within in the enclosed volume due to the consumption of the oxygen, which makes up about 20% of air.

The reactor 90 may be put into fluid communication with the enclosed volume so as to consume (react with) the selected gas from the enclosed volume. The selected gas may be oxygen, and thus when the reactor 90 is used and the valve assembly 20 seals off the first aperture 14, the reactor 90 may generate a low-oxygen environment in the enclosed volume and around the tissue site.

The reactor 90 may be put into fluid communication with the enclosed volume via the second aperture 16 (FIGS. 10-17), wherein the reactor 90 is external to the enclosed volume or arranged in the enclosed volume. In a non-limiting embodiment where the reactor 90 is external to the enclosed volume (FIGS. 10-13), the second aperture 16 may be sealed in a substantially air-tight manner, e.g. by a cap assembly 92. The cap assembly 92 may include a base 94 sealed to the drape and around the second aperture 16 in a substantially air-tight manner, e.g. by an adhesive, and a cap 96 that selectively attaches to the base 94 (e.g. by threads) to seal the opening 98 in the base 94 in a substantially air-tight manner.

The reactor 90 may be arranged in the cap 96 (i.e. the reactor 90 is included as part of the cap 96), and may be protected from exposure to the selected gas by a protective liner 100. The protective liner 100 may prevent the selected gas from reaching the reactor 90, and in this state the reactor 90 is considered not activated. To activate the reactor 90, the protective liner 100 may be removed from the cap 96 or a seal 102 of the protective liner 100 may be broken. With reference to FIGS. 12-13, a seal 102 of the protective liner 100 is broken by rotating the cap 96 with respect to the base 94 as shown in FIG. 10. Such rotation of the cap 96 causes the seal 102 to be pressed down onto a side wall 104 of the base 94, which may break the seal 102 and thus expose the reactor 90 to the selected gas. Because the reactor 90 is in fluid communication with the enclosure volume through the second aperture 16, the reactor 90 reacts with the selected gas in the enclosed volume by consuming the selected gas. With reference to FIG. 11, the protective liner 100 may be peeled away from the reactor 90, which may lie underneath the protective liner 100 in FIG. 11. The protective liner 100 may include a finger tab 108 for pulling the protective liner 100 off of the reactor 90, thus exposing the reactor 90 to the selected gas. The cap 96 is then screwed onto the base 94 as shown in FIG. 10, thus putting the reactor 90 into fluid communication with the enclosure volume through the second aperture 16 and allowing the reactor 90 to consume the selected gas in the enclosed volume. In any of FIGS. 10-13, the cap 96 may be replaced when the reactor 90 is used up by reaction with oxygen and another reactor is needed.

The reactor 90 may also be arranged within the enclosed volume (FIGS. 14-17) and activated via the second aperture 16. In FIGS. 14-17, the second aperture 16 is a slit in the drape 4, through which the protective liner 100 extends. The reactor 90 is arranged inside the enclosed volume, e.g. attached to an underside of the drape 4. A tab 108 of the protective liner 100 may be pulled, which results in the protective liner 100 being removed from covering the reactor 90 and thus exposing the reactor 90 to the selected gas in the enclosed volume. The protective liner 100 can be pulled all the way out of the enclosed volume through the second aperture 16 and discarded. A second tab 110 can be attached to a second protective liner 106. The second tab 110 can then be pulled to release from the second protective liner 106 and allow the second protective liner 106 to adhere to the top surface of the drape 4 and over the second aperture 16 to thus seal the second aperture 16, thus sealing the enclosed volume at the second aperture 16.

The reactor 90 can be one, for example, as described in US 2014/0109890 A1 and/or the oxygen absorption means described in U.S. Pat. No. 8,012,169 B2, which are incorporated by reference herein. US 2014/0109890 A1 describes an air-activated heater; however, the air-activated heater described in US 2014/0109890 A1 can be used as the reactor 90 to consume oxygen within the enclosed volume, thus producing a partial vacuum within the enclosed volume. The reactor 90 may include a reducing agent, a binding agent on a reactor substrate, or an electrolyte solution, which can be provided in an electrolyte impregnated pad. The reducing agent on the reactor substrate can be zinc, aluminum, or iron, for example.

In the subject dressing 2, the drape 4 may be flexible, and thus may collapse toward the tissue site when air is removed by the mechanical pump assembly 86 and/or when the selected gas is consumed by the reactor 90. In which case, the volume of air/gas between the tissue site and the drape 4 may decrease proportionally as the air/gas is removed, thus maintaining a total gas pressure in the enclosed volume that is close to the ambient air pressure in accordance with the ideal gas law (PV=nRT). Initially, PₐₜₘVᵢₙᵢₜᵢₐₗ=nᵢₙᵢₜᵢₐₗRT, or Pₐₜₘ Vᵢₙᵢₜᵢₐₗ=(n_{N2}+n_{O2})RT. To arrive at the final condition with P=Pₐₜₘ according to PₐₜₘV_{final}=n_{N2}RT, V_{final}/Vᵢₙᵢₜᵢₐₗ must=n_{N2}/(n_{N2}+n_{O2}). In other words, if a volume beneath the drape 4 is not maintained, such as when the flexible drape 4 collapses, then negative pressure (with respect to atmospheric pressure) is not produced in the enclosed volume and instead, atmospheric pressure is maintained in the enclosed volume. Even if the total gas pressure within the enclosed volume is near or equal with atmospheric pressure however, a low-oxygen environment can still be achieved beneath the drape 4 since the reactor 90 is removing the selected gas (i.e. oxygen) from the enclosed volume. If the drape 4 is instead rigid or if the dressing 2 includes internal components that are resistant to compression, then removal of air by the mechanical pump assembly 86 and/or removal of the selected gas by the reactor 90 may not cause the drape 4 to collapse, and thus the total gas pressure in the enclosed volume may decrease below atmospheric pressure. In the case of using the reactor 90, the amount of oxygen in the enclosed volume may also be reduced, thus also producing a low-oxygen environment.

It will be appreciated that various features of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A device for tissue treatment comprising:
a drape configured to seal to skin of a patient to define an air-tight enclosed volume under the drape and around a tissue site, the drape including an aperture extending through the drape from a top surface to a bottom surface of the drape and thus providing access to the enclosed volume; and
a valve assembly attached to the drape, covering the aperture, and regulating a bulk flow of air through the aperture, the valve assembly including:
a base,
a top that is moveable with respect to the base between an opened position allowing the bulk flow of air through the aperture, and a closed position preventing the bulk flow of air through the aperture, and
a seal arranged on the top, and sealing to the base over the aperture when the top is in the closed position.

2. The device according to claim 1, wherein:
the top is connected to the base by a hinge joint;
the top is moveable between the opened position and the closed position by rotating the top relative to the base about the hinge joint;
the top includes a first annular projection that aligns the top to the base by fitting radially inside a second annular projection on the base when the top is in the closed position;
the seal is an annular seal arranged radially inside the first annular projection; and
when the top is in the closed position, the annular seal seals to an upper surface of the base radially outside a through hole in the base.

3. The device according to claim 1, wherein:
the top is connected to the base by a threaded joint;
the top is moveable between an opened position and a closed position by rotating the top relative to the base; and
the seal includes a first annular seal and a second annular seal; and
when the top is in the closed position, the first annular seal seals to an upper surface of the base radially outside a through hole in the base, and the second annular seal seals to the upper surface of the base radially inside the through hole.

4. The device according to claim 3, wherein:
the base includes a projection; and
when the top is in the closed position, the projection is elevated above the top to provide a visual indication of the closed position.

5. The device according to claim 1, wherein:
the top is connected to the base by a slide joint;
the top is moveable between an opened position and a closed position by sliding the top relative to the base;
the seal is an annular seal; and
when the top is in the closed position, the annular seal is arranged is a circular depression in an upper surface of the base and seals to the upper surface of the base radially outside a through hole in the base; and
when the top is in the opened position, the annular seal is arranged at least partially outside the circular depression.

6. The device according to claim 5, wherein:
the slide joint includes a lateral projection on the top slidably arranged in a lateral indentation in the base; and
the lateral indentation are sloped such that the top gets closer to the base as the top is moved from the open position towards the closed position.

7. The device according to claim 1, wherein the valve assembly further includes a valve arranged in the base and configured to open to allow the bulk flow of air through the aperture, and to close to prevent the bulk flow of air through the aperture.

8. The device according to claim 7, wherein the valve opens upon being subject to a pressure condition.

9. The device according to claim 7, wherein:
the base includes a lower base portion and an upper base portion; and
the valve is clamped between the lower base portion and the upper base portion.

10. The device according to claim 9, wherein the through hole in the base is defined by a first through hole in the lower base portion and a second through hole in the upper base portion.

11. The device according to claim 10, wherein the first through hole and the second through hole are aligned over the aperture in the drape.

12. The device according to claim 1, wherein:
the device further includes an adhesive and an adhesive tape;
the adhesive adheres the valve assembly to the top surface of the drape around the aperture, and thus seals the valve assembly to the drape; and
the adhesive tape is adhered to the top surface of the drape and to a lower edge of the base, and thus seals the lower edge of the base to the drape.

13. The device according to claim 1, wherein:
when the top is in the closed position, the top covers the upper surface of the base; and
when the top is in the open position, a portion of the upper surface of the base is not covered by the top.

14. The device according to claim 1, further including an annular ridge extending up from the upper surface of the base, the annular ridge mating with an annular depression in a bottom surface of the seal when the top is in the closed position.

15. The device according to claim 1, wherein:
the aperture is a first aperture;
the drape further includes a second aperture extending through the drape from the top surface to the bottom surface and thus providing access to the enclosed volume; and
the device further includes a reactor in fluid communication with the enclosed volume and that when activated reacts with a selected gas found in air, thus consuming the selected gas in the enclosed volume.

16. The device according to claim 15, wherein:
the device further includes a cap assembly selectively sealing the second aperture in an air-tight manner;
the cap assembly includes a cap and a cap base;
the cap base is sealed to the top surface of the drape;
the cap is selectively connected to the cap base to seal the second aperture in an air-tight manner and thus regulate a bulk flow of air through the second aperture;
the reactor is included as part of the cap;
the reactor is activated by connecting the cap to the cap base, or the reactor is activated by removing a protective liner from the reactor; and
the reactor is in fluid communication with the enclosed volume through the second aperture when the cap is connected with the cap base.

17. The device according to claim 15, wherein:
the reactor is arranged in the enclosed volume and is activated by removing a first protective liner from the reactor by pulling the first protective liner through the second aperture; and
the device further includes a second protective liner selectively sealing the second aperture in an air-tight manner after the reactor is activated.

18. A system for tissue treatment comprising:
the device according to claim 1; and
a mechanical pump that when activated and engaged to the upper surface of the base when the top is in the opened position, thereby removes air from the enclosed volume.

19. The system according to claim 18, wherein when the mechanical pump is engaged to the upper surface of the top and is activated, the valve opens to thereby allow air to flow out of the enclosed volume through the aperture.

20. The system according to claim 18 or 19, wherein the valve is biased to be closed.
